(19) United States
(12) Patent Application Publication
(10) Pub. No.: US 2010/0297588 A1
(43) Pub. Date: Nov. 25, 2010

(54) DENTAL COMPOSITIONS WITH NATURAL TOOTH FLUORESCENCE AND METHODS

(75) Inventors: Rajdeep S. Kalgutkar, Woodbury, MN (US); Gregory A. Kobussen, Woodbury, MN (US)

Correspondence Address: 3M INNOVATIVE PROPERTIES COMPANY, PO BOX 33427, ST. PAUL, MN, 55133-3427, US

(73) Assignee: 3M INNOVATIVE PROPERTIES COMPANY, St. Paul, MN (US)

(21) Appl. No.: 12/740,528

(22) PCT Filed: Oct. 29, 2008
(86) PCT No.: PCT/US2008/081573

§ 371 Date: Apr. 29, 2010

Related U.S. Application Data
(60) Provisional application No. 60/984,785, filed on Nov. 2, 2007.

(51) Int. Cl. A61C 5/00; A61K 6/08

(52) U.S. Cl. 433/228.1; 523/116

(57) ABSTRACT

Hardenable dental compositions which include a fluorescent diether terephthalate and a resin system, such that when hardened, the compositions have a natural tooth fluorescence, methods of making the compositions, and dental articles made from the compositions are disclosed.

### CROSS REFERENCE TO RELATED APPLICATIONS

The present invention claims priority to U.S. Provisional Application Ser. No. 60/984,785, filed Nov. 2, 2007, which is incorporated herein by reference.

### BACKGROUND

Over the past several decades, there has been an increasing demand among dentists and dental patients for more aesthetic dental restorations. High aesthetic quality is desirable for restorations involving the anterior teeth, but may also be desirable for restorations involving teeth that are not as readily visible.

The dental industry's growing focus on aesthetic dentistry has led to the development of dental restorative compositions that more closely mimic the appearance of natural teeth. For example, tooth-colored, composite resin materials have been developed that can be used in place of, for example, metal amalgam fillings, to provide more natural looking dental restorations. In recent years, highly aesthetic composite materials, such as 3M ESPE™ FILTEK™ Supreme Plus Universal Restorative (3M Company, St. Paul, Minn.), have become available with shading systems and opacity options that make it possible for a dentist to create dental restorations so natural looking they are virtually undetectable to the casual observer.

Since human teeth fluoresce when irradiated with ultraviolet (UV) light, dental restorations that fail to exhibit fluorescence similar to that of natural teeth may become more noticeable when viewed under UV radiation or “black light” conditions. For example, dental restorative compositions that use resin systems that do not fluoresce as intensely as natural teeth and/or that contain components, such as color stabilizers and shading pigments, that diminish the fluorescence of the composition, may provide restorations that appear darker than surrounding teeth under UV light. Conversely, dental compositions that contain components with greater fluorescence than that of natural teeth may appear brighter than surrounding teeth under these conditions. Consequently, restorations made with such compositions, even if undetectable under normal visible light or full spectrum lighting conditions, may suffer from reduced aesthetic quality when exposed to UV light.

Dental compositions having natural tooth fluorescence are, therefore, desirable, and there continues to be interest in such compositions.

### SUMMARY

The present invention provides new hardenable dental compositions which when hardened have natural tooth fluorescence. Such compositions comprise a fluorescent compound of the Formula I:

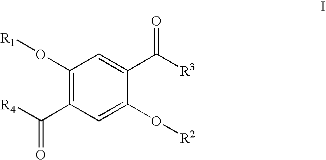

wherein R₁, R₂, R₃, and R₄ are defined below, and a resin system; wherein the composition upon hardening has a natural tooth fluorescence. These compositions are useful for making aesthetic dental articles, for example, restoratives, that have a natural-looking appearance even when viewed under ultraviolet light or black light.

The invention further provides a dental article made by hardening such hardenable dental compositions.

The invention also provides a method of making a dental article, the method comprising the steps of providing a hardenable dental composition described above or any one of the embodiments thereof described below; and hardening the composition.

The invention still further provides a method of making a hardenable dental composition which upon hardening has natural tooth fluorescence, the method comprising the steps of providing a hardenable material that, upon hardening, provides a material that has a non-natural tooth fluorescence; and adding a sufficient amount of a fluorescent compound of Formula I to the hardenable material such that, upon hardening, the material has a natural tooth fluorescence; wherein the fluorescent compound of Formula I is:

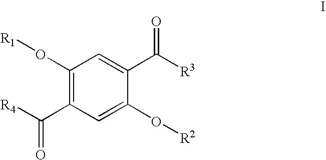

wherein R₁, R₂, R₃, and R₄ are defined below; and wherein the hardenable material includes a resin system.

The hardenable dental compositions of the invention are useful in a variety of dental and orthodontic applications, including, for example, dental restoratives, dental adhesives, dental cements, cavity liners, orthodontic adhesives, dental sealants, dental coatings, and the like. The compositions and related methods may be used to prepare dental articles by hardening to form, for example, dental fillings, dental mill blanks, dental crowns, dental prostheses, orthodontic devices, and the like.

The above summary of the present invention is not intended to describe each disclosed embodiment or every implementation of the present invention. The description that follows more particularly exemplifies illustrative embodiments. In several places throughout the description, guidance is provided through lists of examples, which examples can be used in various combinations. In each instance, the recited list serves only as a representative group and should not be interpreted as an exclusive list.

### DEFINITIONS

As used herein, the terms “alkyl,” “alkenyl,” “alkynyl” and the prefix “alk-” are inclusive of both straight chain and branched chain groups and of cyclic groups, e.g., cycloalkyl and cycloalkenyl. Unless otherwise specified, these groups contain from 1 to 20 carbon atoms, with alkenyl groups containing from 2 to 20 carbon atoms, and alkynyl groups containing from 2 to 20 carbon atoms. In some embodiments, these groups have a total of up to 10 carbon atoms, up to 8 carbon atoms, up to 6 carbon atoms, up to 4 carbon atoms, up to 2 carbon atoms, excluding the cyclic groups, or up to 1 carbon atom, excluding the cyclic, alkenyl, and alkynyl groups. Cyclic groups can be monocyclic or polycyclic and preferably have from 3 to 10 ring carbon atoms. Exemplary cyclic groups include cyclopropyl, cyclopropylmethyl, cyclopentyl, cyclohexyl, adamantyl, and substituted and unsubstituted bornyl, norbornyl, and norbornenyl.

Unless otherwise specified, “alkylene”, “alkenylene”, and “alkynylene” are the divalent forms of the “alkyl”, “alkenyl”, and “alkynyl” groups defined above. The terms, “alkylenyl”, “alkenylenyl”, and “alkynylenyl” are used when “alkylene”, “alkenylene”, and “alkynylene”, respectively, are substituted. For example, a phenylalkylenyl group comprises an alkylene moiety to which a phenyl group is attached.

Unless otherwise indicated, the term “halogen” refers to a halogen atom or one or more halogen atoms.

The term “haloalkyl” is inclusive of groups that are substituted by one or more halogen atoms, including perfluorinated groups. This is also true of other groups that include the prefix “halo-.” Examples of suitable haloalkyl groups are chloromethyl, trifluoromethyl, and the like.

Unless otherwise indicated, the term “heteroatom” refers to the atoms O, S, or N.

The term “heteroaryl” includes aromatic rings or ring systems that contain at least one ring heteroatom (e.g., O, S, N). In some embodiments, the term “heteroaryl” includes a ring or ring system that contains 2-12 carbon atoms, 1-3 rings, 1-4 heteroatoms, and O, S, and N as the heteroatoms. In some embodiments, the term “heteroaryl” includes one ring that contains 2-5 carbon atoms, 1-3 heteroatoms, and O, S, and N as the heteroatoms. Exemplary heteroaryl groups include furyl, thienyl, pyridyl, quinolinyl, isoquinolinyl, indolyl, isoindolyl, triazolyl, pyrrolyl, tetrazolyl, imidazolyl, pyrazolyl, oxazolyl, thiazolyl, benzofuranyl, benzothiophenyl, carbazolyl, benzoxazolyl, pyrimidinyl, benzimidazolyl, quinoxalinyl, benzothiazolyl, naphthyridinyl, isoxazolyl, isothiazolyl, purinyl, quinazolinyl, pyrazinyl, 1-oxidopyridyl, pyridazinyl, triazinyl, tetrazinyl, oxadiazolyl, thiadiazolyl, and so on.

The term “heterocyclyl” includes non-aromatic rings or ring systems that contain at least one ring heteroatom (e.g., O, S, N) and includes all of the fully saturated and partially unsaturated derivatives of the above mentioned heteroaryl groups. In some embodiments, the term “heterocyclyl” includes a ring or ring system that contains 2-12 carbon atoms, 1-3 rings, 1-4 heteroatoms, and O, S, and N as the heteroatoms. In some embodiments, the term “heterocyclyl” includes one ring that contains 2-5 carbon atoms, 1-3 heteroatoms, and O, S, and N as the heteroatoms. Exemplary heterocyclyl groups include pyrrolidinyl, tetrahydrofuranyl, morpholinyl, thiomorpholinyl, 1,1-dioxothiomorpholinyl, piperidinyl, piperazinyl, thiazolidinyl, imidazolidinyl, isothiazolidinyl, tetrahydropyranyl, quinuclidinyl, homopiperidinyl (azepanyl), 1,4-oxazepanyl, homopiperazinyl (diazepanyl), 1,3-dioxolanyl, aziridinyl, azetidinyl, dihydroisoquinolin-(1H)-yl, octahydroisoquinolin-(1H)-yl, dihydroquinolin-(2H)-yl, octahydroquinolin-(2H)-yl, dihydro-1H-imidazolyl, 3-azabicyclo[3.2.2]non-3-yl, and the like.

The term “heterocyclyl” includes bicyclic and tricyclic heterocyclic ring systems. Such ring systems include fused and/or bridged rings and spiro rings. Fused rings can include, in addition to a saturated or partially saturated ring, an aromatic ring, for example, a benzene ring. Spiro rings include two rings joined by one spiro atom and three rings joined by two spiro atoms.

When “heterocyclyl” contains a nitrogen atom, the point of attachment of the heterocyclyl group may be the nitrogen atom.

As used herein, the phrase “natural tooth fluorescence” means that when viewed under ultraviolet light the composition when hardened exhibits a fluorescence having an intensity and range of wavelengths resembling that of a natural tooth. Because the fluorescence of natural teeth varies from subject to subject and the desired closeness of the match of the composition's fluorescence to that of a natural tooth depends on the precise situation and/or aesthetic demands of the patient (e.g., molars and other teeth that are not easily visible may not need to match the natural tooth fluorescence as closely as front teeth), “natural tooth fluorescence” necessarily encompasses a range of intensities and wavelengths. Fluorescence corresponding to “natural tooth fluorescence” may be characterized in terms of CIE 2° Chromaticity Coordinates, x and y, which correspond to perceived color of fluorescence. For certain embodiments, compositions of the invention when hardened exhibit a fluorescence wherein x is in the range of 0.13 to 0.19, and y is in the range of 0.05 to 0.22, when measured using the test methods described herein.

By “non-natural tooth fluorescence” is meant fluorescence that is visibly less intense or more intense than the fluorescence exhibited by natural teeth, and/or fluorescence having a visibly different color range or CIE 2° Chromaticity Coordinates range than that of natural teeth.

By “non-fluorescent” is meant that when irradiated with ultraviolet light, the compound, composition, or material exhibits no visible fluorescence or is only weakly fluorescent, i.e. substantially below the fluorescence exhibited by a natural human tooth such that the difference is easily visible.

As used herein, “hardenable” is descriptive of a material or composition that can be cured (e.g., polymerized or crosslinked) or solidified, for example, by removing solvent (e.g., by evaporation and/or heating); by changing state, such as by crystallizing; heating to induce polymerization and/or crosslinking; irradiating to induce polymerization and/or crosslinking; and/or by mixing one or more components to induce polymerization and/or crosslinking

By “dental composition” is meant an unfilled or filled (e.g. a composite) material (e.g., a dental or orthodontic material) that can be applied or adhered to an oral surface. Dental compositions include, for example, adhesives (e.g., dental and/or orthodontic adhesives), cements (e.g., glass ionomer cements, resin-modified glass ionomer cements, and/or orthodontic cements), primers (e.g., orthodontic primers), restoratives (e.g., a restorative filling material), liners, sealants (e.g., orthodontic sealants), and coatings. In certain embodiments, the dental composition can be used to bond a dental article to a tooth structure.

By “hardenable dental composition” is meant a dental composition, such as a paste, that can be hardened to form a dental article and/or to bond a dental article to a tooth structure.

By “dental article” is meant an article that can be adhered (e.g., bonded) to an oral surface (e.g., a tooth structure). Examples include restoratives, replacements, inlays, onlays, veneers, full and partial crowns, malleable temporary crowns, bridges, implants, implant abutments, copings, anterior fillings, posterior fillings, cavity liners, sealants, dentures, posts, bridge frameworks and other bridge structures, abutments, orthodontic appliances and devices, and prostheses (e.g., partial or full dentures). For certain embodiments, the dental article is a restored dentition or a portion thereof.

As used herein, the terms “dental composition” and “dental article” are not limited to compositions and articles used in dental applications, but also include orthodontic compositions (e.g., orthodontic adhesives) and orthodontic devices (e.g., orthodontic appliances such as retainers, night guards, brackets, buccal tubes, bands, cleats, buttons, lingual retainers, bite openers, positioners, and the like), respectively.

By “oral surface” is meant a soft or hard surface in the oral environment. Hard surfaces typically include tooth structure including, for example, natural and artificial tooth surfaces, bone, tooth models, dentin, enamel, cementum, and the like.

By “filler” is meant a particulate material suitable for use in the oral environment. Dental fillers generally have an average particle size of at most 100 micrometers.

By “nanofiller” is meant a filler having an average primary particle size of at most 200 nanometers. The nanofiller component may be a single nanofiller or a combination of nanofillers. Typically the nanofiller comprises non-pyrogenic nanoparticles or nanoclusters.

By “nanostructured” is meant a material in a form having at least one dimension that is, on average, at most 200 nanometers (e.g., nanosized particles). Thus, nanostructured materials refer to materials including, for example, nanoparticles as defined herein below; aggregates of nanoparticles; materials coated on particles, wherein the coatings have an average thickness of at most 200 nanometers; materials coated on aggregates of particles, wherein the coatings have an average thickness of at most 200 nanometers; materials infiltrated in porous structures having an average pore size of at most 200 nanometers; and combinations thereof. Porous structures include, for example, porous particles, porous aggregates of particles, porous coatings, and combinations thereof.

As used herein “nanoparticles” is synonymous with “nanosized particles,” and refers to particles having an average size of at most 200 nanometers. As used herein for a spherical particle, “size” refers to the diameter of the particle. As used herein for a non-spherical particle, “size” refers to the longest dimension of the particle. In certain embodiments, the nanoparticles are comprised of discrete, non-aggregated and non-agglomerated particles.

By “nanocluster” is meant an association of nanoparticles drawn together by relatively weak intermolecular forces that cause them to clump together, i.e. to aggregate. Typically, nanoclusters have an average size of at most 10 micrometers.

As used herein, the term “ethylenically unsaturated compound” is meant to include monomers, oligomers, and polymers having at least one ethylenic unsaturation.

By “polymerization” is meant the forming of a material having a higher weight from monomer(s) or oligomer(s). The polymerization reaction also can involve a cross-linking reaction.

As used herein, the term “(meth)acrylate” is a shorthand reference to acrylate, methacrylate, or combinations thereof, and “(meth)acrylic” is a shorthand reference to acrylic, methacrylic, or combinations thereof. As used herein, “(meth)acrylate-functional compounds” are compounds that include, among other things, a (meth)acrylate moiety.

The terms “comprises”, “comprising” and variations thereof do not have a limiting meaning where these terms appear in the description and claims.

The recitation herein of numerical ranges by endpoints is intended to include all numbers subsumed within that range (e.g. 1 to 5 includes 1, 1.5, 2, 2.75, 3, 3.80, 4, and 5).

As used herein, “a” or “an” means “at least one” or “one or more” unless otherwise indicated. In addition, the singular forms “a”, “an”, and “the” include plural referents unless the content clearly dictates otherwise. Thus, for example, reference to a composition containing “a compound” includes a mixture of two or more compounds. As used in this specification and the appended claims, the term “or” is generally employed in its sense including “and/or” unless the content clearly dictates otherwise.

Unless otherwise indicated, all numbers expressing quantities of ingredients, measurement of properties and so forth used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings of the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviations found in their respective testing measurements.

### DETAILED DESCRIPTION OF ILLUSTRATIVE EMBODIMENTS OF THE INVENTION

The present invention provides hardenable dental compositions which include a fluorescent diether terephthalate of the Formula I described herein, such that when hardened, the compositions have a natural tooth fluorescence.

In one embodiment, the present invention provides a hardenable dental composition comprising:

a fluorescent compound of the Formula I:

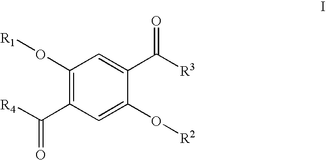

wherein R₁ and R₂ are independently selected from the group consisting of alkyl, alkenyl, alkynyl, phenyl, heteroaryl, and heterocyclyl; each of which is unsubstituted or substituted with one or more substituents independently selected from the group consisting of alkyl, alkenyl, alkoxy, haloalkyl, haloalkoxy, halogen, hydroxy, mercapto, cyano, carboxy, alkoxycarbonyl, alkyloyloxy, acryloyloxy, methacryloyloxy, alkoxyalkylene, glycidoxy, formyl, phenyl, phenoxy, phenylalkoxy, phosphono, dialkylphosphono, phosphonooxy, dialkylphosphonooxy, alkylsulfonyl, phenylsulfonyl, heteroaryl, heteroaryloxy, heteroarylalkoxy, heterocyclyl, heterocyclylalkylenyl, and in the case of alkyl, alkenyl, alkynyl, and heterocyclyl, oxo and epoxy; R₃ and R₄ are independently selected from the group consisting of HO—, alkyl-O—, (meth)acryloyl-O-alkylene-O—, phenyl-O—, heteroaryl-O—, and M⁺⁻O—; wherein M⁺ is a monovalent metal cation; and

a resin system; wherein the composition upon hardening has a natural tooth fluorescence.

In another embodiment, there is provided a method of making a hardenable dental composition which upon hardening has natural tooth fluorescence, the method comprising the steps of providing a hardenable material that, upon hardening, provides a material that has a non-natural tooth fluorescence; and adding a sufficient amount of a fluorescent compound of Formula I to the hardenable material such that, upon hardening, the material has a natural tooth fluorescence; wherein the fluorescent compound of Formula I is as described above; and wherein the hardenable material includes a resin system.

For certain embodiments, including the above embodiments, R₁ and R₂ are each independently alkyl, acryloyloxyalkylenyl, methacryloyloxyalkylenyl, glycidoxyalkylenyl, or glycidoxyphenyl. For certain of these embodiments, R₁ and R₂ are each independently alkyl.

For certain embodiments, including any one of the above embodiments, R₃ and R₄ are each independently alkoxy.

For certain of these embodiments, including any one of the above embodiments, R₁ and R₂ are methyl and R₃ and R₄ are ethoxy.

Compounds of Formula I are highly fluorescent, have good light stability, and are soluble in the resin system. The R₁ and R₂ groups can be varied to control the fluorescence maximum to some extent. In addition, these groups may include ethylenically unsaturated groups, epoxy groups, and/or other polymerizable or crosslinkable groups, which allow the fluorescent compound to be copolymerized with the resin system. This provides the advantage that the fluorescent compound is prevented from leaching out of the hardened composition.

For certain embodiments, R₁ and R₂ are each independently acryloyloxyalkylenyl, methacryloyloxyalkylenyl, glycidoxyalkylenyl, or glycidoxyphenyl.

For certain embodiments, including any one of the above embodiments, the fluorescence from the composition upon hardening has chromaticity coordinates x and y, wherein x is in the range of 0.13 to 0.19, and y is in the range of 0.05 to 0.22. For certain of these embodiments, x is in the range of 0.14 to 0.18, and y is in the range of 0.09 to 0.20. The chromaticity coordinates, which are CIE 2° Chromaticity Coordinates, correspond to the perceived color of the fluorescence, and are obtained as described under Fluorescence Test Method in the Example section below.

For certain embodiments, including any one of the above embodiments, the fluorescence, from the composition upon hardening, has a wavelength of maximum emission, wherein the wavelength is in the range of 410 nm to 475 nm. For certain of these embodiments, the wavelength is in the range of 420 nm to 460 nm. Fluorescence within these ranges has a blue-white appearance, which typically is the appearance of natural tooth fluorescence.

For certain embodiments, including any one of the above embodiments, the degree of yellowness of the composition, as expressed by the color coordinate b*, is not more than 15. This degree of yellowness is determined in the absence of pigments used to shade or color the composition and prior to aging or color stability testing.

For certain embodiments, including any one of the above embodiments, the hardenable dental composition further comprises at least one filler. The presence of a filler can affect certain properties of hardened dental compositions, for example, appearance, radiopacity, and physical and mechanical properties. The appearance of a dental material can be made to closely approximate the appearance of natural dentition according to the ingredients, including the at least one filler, of the composition. Suitable fillers are described herein below. For certain of these embodiments which include at least one filler, the at least one filler includes zirconia. For certain of these embodiments, the at least one filler includes an aggregate of nanozirconia and nanosilica (zirconia-silica nanoclusters). The use of zirconia or nanozirconia can contribute strength, durability, and abrasion resistance to the hardened compositions, and in the case of nanozirconia, increased translucency can be additionally provided. However, it has now been found that compositions containing zirconia-silica nanoclusters have a deep yellow color in the presence of diethyl 2,5-dihydroxyterephthalate, a fluorescent dye having a fluorescence maximum which closely matches that of natural dentin and enamel. The diethyl 2,5-dihydroxyterephthalate dye has been found to cause yellowing of compositions, even at low levels of this dye in the presence of zirconia-silica nanoclusters. It is believed that such yellowing may occur in the presence of zirconia in various forms. Moreover, the diethyl 2,5-dihydroxyterephthalate dye itself is yellow and can, therefore, cause yellowing directly even in the absence of any form of zirconia, although the extent of yellowing is much less. In contrast, the compounds of Formula I have been found to provide a natural tooth fluorescence, even in the presence of zirconia-silica nanoclusters, without causing the composition to turn deep yellow. Moreover, compounds of Formula I, such as diethyl 2,5-dimethoxyterephthalate, can be obtained as a colorless solid, and in certain embodiments, these compounds do not, on their own, cause the composition to be yellow.

For certain embodiments, including any one of the above embodiments, the fluorescent compound of the Formula I is present in an amount of 0.0001 to 0.5 weight percent based upon the weight of the composition. For certain of these embodiments, the fluorescent compound of the Formula I is present in an amount of 0.001 to 0.10 weight percent based upon the weight of the composition.

For certain embodiments, including any one of the above embodiments, except those where the weight percent is based upon the weight of the composition, the fluorescent compound of the Formula I is present in an amount of 0.01 to 1.0 weight percent based upon the weight of the resin system. For certain of these embodiments, the fluorescent compound of the Formula I is present in an amount of 0.01 to 0.5 weight percent based upon the weight of the resin system.

The resin system may include a material which contributes to or provides for the hardenability of the composition. In one example, this material may be a polymer, which can be dissolved in a solvent, such that when the solvent is removed, the composition is hardened. Such polymers include, for example, poly(meth)acrylates. In another example, this material may be a crystallizable compound, such that when crystallized, the composition is hardened. Such crystallizable compounds include, for example, thermoplastics, for example, polylactones. In another example, this material may be a polymerizable resin, such that when the material undergoes polymerization, the composition is hardened. The term “polymerizable” includes crosslinkable. Polymerization and/or crosslinking can be carried out by applying heat, by applying actinic radiation (photopolymerizing), and/or by mixing one or more components with the polymerizable resin to induce polymerization and/or crosslinking Suitable polymerizable resins are described herein below.

For certain embodiments, including any one of the above embodiments, the resin system comprises a polymerizable resin.

The resin system may further include an initiator system which can be used in conjunction with the polymerizable resin. Suitable initiator systems are described herein below.

For certain embodiments, including any one of the above embodiments, the resin system comprises a polymerizable resin and an initiator system. For certain of these embodiments, the polymerizable resin comprises an ethylenically unsaturated compound, an epoxy compound, or a combination thereof. For certain of these embodiments, the polymerizable resin comprises an ethylenically unsaturated compound. For certain of these embodiments, the ethylenically unsaturated compound is a (meth)acrylate. Alternatively, for certain of these embodiments which include a polymerizable resin, the polymerizable resin comprises an epoxy compound.

As indicated above, natural tooth fluorescence varies from subject to subject. Accordingly, customizing the fluorescence of the present compositions may be desired to even more closely approximate the natural tooth fluorescence of a particular subject. This may be carried out by adjusting the amount of the fluorescent compound of Formula I, by using one or a combination of fluorescent compounds of Formula I having R₁ and R₂ groups which provide for a desired fluorescence, and/or by using at least one additional fluorescent material, such as a luminescent organic dye or an inorganic phosphor, in the hardenable dental compositions. For certain embodiments, including any one of the above embodiments, the hardenable composition or the hardenable material further comprises at least one additional fluorescent material having a maximum wavelength of emission in the range of 400 nm to 500 nm. For certain embodiments, the range of 440 to 480 nm is preferred. For certain embodiments, the range of 450 nm to 470 nm is preferred. The additional fluorescent material preferably does not cause the composition to be a deep yellow, whether or not a zirconia-silica nanocluster is present. For certain embodiments, the additional fluorescent material has no significant absorbance of light having a wavelength greater than 420 nm, preferably greater than 400 nm, more preferably greater than 390 nm.

Examples of additional fluorescent materials that may be used include derivatives of pyrazoline, stilbene, triazine, thiazole, benzoxazole, xanthone, triazole, oxazole, thiophene, and coumarin. Other examples may be found in U.S. Pat. Nos. 6,933,327 (Yamakawa et al.) at column 12, lines 38 to 49; 7,114,951 (Sun et al.) at column 2, lines 28 to 45; 7,137,818 (Savic et al.) at column 2, lines 46 to 54, each of which is incorporated herein by reference; and in “Handbook Of Fluorescence Spectra Of Aromatic Molecules”, Isadore B. Berlman, New York, Academic Press (1971), taking the above described criteria into account.

The additional fluorescent material may be present in an amount of 0.0001 to 0.5 or 0.001 to 0.10 weight percent based upon the weight of the composition.

In another embodiment, the present invention provides a dental article made by hardening a composition, the composition being any one of the above embodiments of the hardenable dental composition.

In another embodiment, the present invention provides a method of making a dental article, the method comprising the steps of providing a hardenable dental composition, the composition being any one of the above embodiments of the hardenable dental composition; and hardening the composition. For certain of these embodiments, hardening the composition is carried out by photopolymerizing the hardenable dental composition.

For certain embodiments, including any one of the above embodiments which includes a dental article, the dental article is selected from the group consisting of restoratives, replacements, inlays, onlays, veneers, full and partial crowns, malleable temporary crowns, bridges, implants, implant abutments, copings, anterior fillings, posterior fillings, cavity liners, sealants, dentures, posts, bridge frameworks, abutments, and orthodontic appliances.

### Polymerizable Resins

The dental compositions of the present invention are hardenable, and in certain embodiments, this is based upon the presence of a polymerizable resin. In some embodiments, the compositions can be hardened (e.g., polymerized by conventional photopolymerization and/or chemical polymerization techniques) prior to being applied to an oral surface. In other embodiments, the compositions can be hardened (e.g., polymerized by conventional photopolymerization and/or chemical polymerization techniques) after being applied to an oral surface.

In certain embodiments, the compositions are photopolymerizable, i.e., the compositions contain a photoinitiator system that upon irradiation with actinic radiation initiates the polymerization (or hardening) of the composition. In other embodiments, the compositions are chemically hardenable, i.e., the compositions contain a chemical initiator (i.e., initiator system) that can polymerize, cure, or otherwise harden the composition without dependence on irradiation with actinic radiation. Such chemically hardenable compositions are sometimes referred to as “self-cure” compositions and may include glass ionomer cements (e.g., conventional and resin-modified glass ionomer cements), redox cure systems, and combinations thereof.

Polymerizable resins include ethylenically unsaturated compounds (which contain free radically active unsaturated groups, e.g., acrylates and methacrylates), epoxy compounds (which contain cationically active epoxy groups), cyclic allylic compounds, vinyl ether compounds (which contain cationically active vinyl ether groups), and combinations thereof. Polymerizable resins can contain both a cationically active functional group and a free radically active functional group in a single compound. Examples include epoxy-functional (meth)acrylates. Such polymerizable resins are photopolymerizable when combined with a photoinitiator or a photoinitiator system, thereby rendering the composition photopolymerizable.

Ethylenically unsaturated compounds include monomers, oligomers, and polymers having ethylenic unsaturation and can further have acid functionality and/or acid-precursor functionality. Acid functionality includes, for example, carboxylic acid functionality, phosphoric acid functionality, phosphonic acid functionality, sulfonic acid functionality, and combinations thereof. Acid-precursor functionalities include, for example, anhydrides, acid halides, and pyrophosphates.

A polymerizable resin which comprises an ethylenically unsaturated compound typically includes one or more ethylenically unsaturated compounds with or without acid functionality. Suitable compounds contain at least one ethylenically unsaturated bond and are capable of undergoing addition polymerization. Examples of useful ethylenically unsaturated compounds include acrylic acid esters, methacrylic acid esters, hydroxy-functional acrylic acid esters, hydroxy-functional methacrylic acid esters, and combinations thereof. More specific examples of such free radically polymerizable compounds include mono-, di- or poly-(meth)acrylates (i.e., acrylates and methacrylates) such as, methyl (meth)acrylate, ethyl acrylate, isopropyl methacrylate, n-hexyl acrylate, stearyl acrylate, allyl acrylate, glycerol triacrylate, ethyleneglycol diacrylate, diethyleneglycol diacrylate, triethyleneglycol dimethacrylate, 1,3-propanediol di(meth)acrylate, trimethylolpropane triacrylate, 1,2,4-butanetriol trimethacrylate, 1,4-cyclohexanediol diacrylate, pentaerythritol tetra(meth)acrylate, sorbitol hexacrylate, tetrahydrofurfuryl (meth)acrylate, bis[1-(2-acryloxy)]-p-ethoxyphenyldimethylmethane, bis[1-(3-acryloxy-2-hydroxy)]-p-propoxyphenyldimethylmethane, ethoxylated bisphenol A di(meth)acrylate, and trishydroxyethyl-isocyanurate trimethacrylate; (meth)acrylamides (i.e., acrylamides and methacrylamides) such as (meth)acrylamide, methylene bis-(meth)acrylamide, and diacetone (meth)acrylamide; urethane (meth)acrylates; the bis-(meth)acrylates of polyethylene glycols (preferably of molecular weight 200-500), copolymerizable mixtures of acrylated monomers such as those in U.S. Pat. No. 4,652,274 (Boettcher et al.), acrylated oligomers such as those of U.S. Pat. No. 4,642,126 (Zador et al.), and poly(ethylenically unsaturated) carbamoyl isocyanurates such as those disclosed in U.S. Pat. No. 4,648,843 (Mitra); and vinyl compounds such as styrene, diallyl phthalate, divinyl succinate, divinyl adipate and divinyl phthalate. Other suitable free radically polymerizable compounds include siloxane-functional (meth)acrylates as disclosed, for example, in WO-00/38619 (Guggenberger et al.), WO-01/92271 (Weinmann et al.), WO-01/07444 (Guggenberger et al.), WO-00/42092 (Guggenberger et al.) and fluoropolymer-functional (meth)acrylates as disclosed, for example, in U.S. Pat. No. 5,076,844 (Fock et al.), U.S. Pat. No. 4,356,296 (Griffith et al.), EP-0373 384 (Wagenknecht et al.), EP-0201 031 (Reiners et al.), and EP-0201 778 (Reiners et al.). Mixtures of two or more free radically polymerizable compounds can be used if desired.

Suitable ethylenically unsaturated compounds comprising the polymerizable resin may also contain hydroxyl groups and ethylenically unsaturated groups in a single molecule. Examples of such materials include hydroxyalkyl (meth)acrylates, such as 2-hydroxyethyl (meth)acrylate and 2-hydroxypropyl (meth)acrylate; glycerol mono- or di-(meth)acrylate; trimethylolpropane mono- or di-(meth)acrylate; pentaerythritol mono-, di-, and tri-(meth)acrylate; sorbitol mono-, di-, tri-, tetra-, or penta-(meth)acrylate; and 2,2-bis[4-(2-hydroxy-3-methacryloxypropoxy)phenyl]propane (bisGMA). Suitable ethylenically unsaturated compounds are also available from a wide variety of commercial sources, such as Sigma-Aldrich, St. Louis. Mixtures of ethylenically unsaturated compounds can be used if desired.

In certain embodiments, the polymerizable resin includes PEGDMA (polyethyleneglycol dimethacrylate having a molecular weight of approximately 400), bisGMA, UDMA (urethane dimethacrylate), GDMA (glycerol dimethacrylate), TEGDMA (triethyleneglycol dimethacrylate), bisEMA6 as described in U.S. Pat. No. 6,030,606 (Holmes), and/or NPGDMA (neopentylglycol dimethacrylate). Various combinations of these hardenable components can be used if desired.

When the composition contains an ethylenically unsaturated compound without acid functionality, it is generally present in an amount of at least 5% by weight, more typically at least 10% by weight, and most typically at least 15% by weight ethylenically unsaturated compounds without acid functionality, based on the total weight of the unfilled composition. The compositions of the present invention typically include at most 95% by weight, more typically at most 90% by weight, and most typically at most 80% by weight ethylenically unsaturated compounds without acid functionality, based on the total weight of the unfilled composition.

In some embodiments, the polymerizable resin may include one or more ethylenically unsaturated compounds with acid functionality. As used herein, ethylenically unsaturated compounds “with acid functionality” is meant to include monomers, oligomers, and polymers having ethylenic unsaturation and acid and/or acid-precursor functionality (as described above).

Ethylenically unsaturated compounds with acid functionality include, for example, α,β-unsaturated acidic compounds such as glycerol phosphate mono(meth)acrylates, glycerol phosphate di(meth)acrylates, hydroxyethyl (meth)acrylate (e.g., HEMA) phosphates, bis((meth)acryloxyethyl) phosphate, ((meth)acryloxypropyl) phosphate, bis((meth)acryloxypropyl) phosphate, bis((meth)acryloxy)propyloxy phosphate, (meth)acryloxyhexyl phosphate, bis((meth)acryloxyhexyl) phosphate, (meth)acryloxyoctyl phosphate, bis((meth)acryloxyoctyl) phosphate, (meth)acryloxydecyl phosphate, bis((meth)acryloxydecyl) phosphate, caprolactone methacrylate phosphate, citric acid di- or tri-methacrylates, poly(meth)acrylated oligomaleic acid, poly(meth)acrylated polymaleic acid, poly(meth)acrylated poly(meth)acrylic acid, poly(meth)acrylated polycarboxyl-polyphosphonic acid, poly(meth)acrylated polychlorophosphoric acid, poly(meth)acrylated polysulfonate, poly(meth)acrylated polyboric acid, and the like, and may be used as components in the hardenable component system. Also monomers, oligomers, and polymers of unsaturated carbonic acids such as (meth)acrylic acids, aromatic (meth)acrylated acids (e.g., methacrylated trimellitic acids), and anhydrides thereof can be used. In certain embodiments, compositions of the present invention preferably include an ethylenically unsaturated compound with acid functionality having at least one P—OH moiety.

Certain of these compounds with acid functionality are obtained, for example, as reaction products between isocyanatoalkyl (meth)acrylates and carboxylic acids. Additional compounds of this type having both acid-functional and ethylenically unsaturated moieties are described in U.S. Pat. Nos. 4,872,936 (Engelbrecht) and 5,130,347 (Mitra). A wide variety of such compounds containing both the ethylenically unsaturated and acid moieties can be used. Mixtures of such compounds can be used if desired.

Additional ethylenically unsaturated compounds with acid functionality include, for example, polymerizable bisphosphonic acids; AA:ITA:IEM (copolymer of acrylic acid:itaconic acid with pendent methacrylate made by reacting AA:ITA copolymer with sufficient 2-isocyanatoethyl methacrylate to convert a portion of the acid groups of the copolymer to pendent methacrylate groups as described, for example, in Example 11 of U.S. Pat. No. 5,130,347 (Mitra)); and those recited in U.S. Pat. Nos. 4,259,075 (Yamauchi et al.), 4,499,251 (Omura et al.), 4,537,940 (Omura et al.), 4,539,382 (Omura et al.), 5,530,038 (Yamamoto et al.), 6,458,868 (Okada et al.), and European Pat. Application Publication Nos. EP 712,622 (Tokuyama Corp.) and EP 1,051,961 (Kuraray Co., Ltd.).

The polymerizable resin may include combinations of ethylenically unsaturated compounds with acid functionality. The polymerizable resin may also include a mixture of ethylenically unsaturated compounds both with and without acid functionality.

When the composition contains an ethylenically unsaturated compound with acid functionality, it is generally present in an amount of at least 1% by weight, more typically at least 3% by weight, and most typically at least 5% by weight ethylenically unsaturated compounds with acid functionality, based on the total weight of the unfilled composition. The compositions of the present invention typically include at most 80% by weight, more typically at most 70% by weight, and most typically at most 60% by weight ethylenically unsaturated compounds with acid functionality, based on the total weight of the unfilled composition.

Epoxy compounds which are suitable for use as polymerizable resins in the present compositions include, for example, cycloaliphatic oxiranes, aliphatic oxiranes, aromatic oxiranes, or a combination thereof. These compounds, which are widely known as epoxy compounds, can be monomeric, polymeric, or mixtures thereof. These materials generally have, on the average, at least one polymerizable epoxy group (oxirane unit) per molecule, and preferably at least about 1.5 polymerizable epoxy groups per molecule. The polymeric epoxides include linear polymers having terminal epoxy groups (e.g., a diglycidyl ether of a polyoxyalkylene glycol), polymers having skeletal oxirane units (e.g., polybutadiene polyepoxide), and polymers having pendent epoxy groups (e.g., a glycidyl methacrylate polymer or copolymer). The epoxides may be pure compounds or may be mixtures containing one, two, or more epoxy groups per molecule. The “average” number of epoxy groups per molecule is determined by dividing the total number of epoxy groups in epoxy-containing material by the total number of epoxy molecules present. The epoxy compounds may have a molecular weight of from about 58 to about 100,000 or more. The epoxy compounds may further include substituent groups that do not substantially interfere with cationic cure at room temperature, such as halogens, ester groups, ethers, sulfonate groups, siloxane groups, nitro groups, phosphate groups, and the like.

Suitable epoxy compounds include those which contain cyclohexene oxide groups, such as the epoxycyclohexanecarboxylates, for example, 3,4-epoxycyclohexylmethyl-3,4-epoxycyclohexanecarboxylate, 3,4-epoxy-2-methylcyclohexylmethyl-3,4-epoxy-2-methylcyclohexane carboxylate, and bis(3,4-epoxy-6-methylcyclohexylmethyl) adipate. A more detailed list of useful epoxides of this nature is provided in U.S. Pat. No. 3,117,099 (Proops et al.), which is incorporated herein by reference.

Suitable epoxy compounds also include glycidyl ether compounds, such as glycidoxyalkyl and glycidoxyaryl compounds containing 1 to 6 glycidoxy groups. Examples include glycidyl ethers of polyhydric phenols, which can be obtained by reacting the polyhydric phenol with an excess of epichlorohydrin to provide, for example, 2,2-bis(2,3-epoxypropoxyphenyl)propane. Additional epoxides of this type are described in U.S. Pat. No. 3,018,262 (Schroeder), which is incorporated herein by reference, and in “Handbook of Epoxy Resins” by Lee and Neville, McGraw-hill Book Co., New York (1967).

Many suitable epoxy compounds are commercially available and are listed in U.S. Pat. No. 6,187,833 (Oxman et al.).

Cyclic allylic compounds which are suitable for use as polymerizable resins in the present compositions include compounds having a 7 to 10 membered ring containing 1 to 3 heteroatoms in the ring (e.g., S, O, N), wherein at least one heteroatom is sulfur, which may be present as —S—, —S(O)—, —S(O)₂—, or —S—S—, and wherein the ring is substituted by a methylene group at a ring carbon atom which is adjacent a ring carbon atom bonded to the sulfur atom. For certain embodiments, the ring includes two sulfur atoms or a sulfur atom and an oxygen atom or a nitrogen atom in the ring. For certain embodiments, preferably the ring is a 7 to 8 membered ring, and the ring includes two sulfur atoms. In addition, the cyclic allylic compound can include two or more of these rings. The cyclic allylic compound can further include at least one (meth)acryloyl group. Such compounds include, for example, 7-methylene-1,5-dithiacyclooctan-3-yl acetate, 1,6-bis(7-methylene-1,5-dithiacyclooctan-3-yl)-2,4,4-trimethylhexane, 8-methylene-5,11-dihydro-6,10-dithia-benzocyclononane, and 1-(2-methacryloyloxyethyl)-2-(7-methylene-1,5-dithiaoctan-3-yl) phthalate.

Further examples of and methods of preparing suitable cyclic allylic compounds for use as a polymerizable resin in the present compositions are described in International Publication Nos. WO 2006/122074 (Abuelyaman et al.) and WO 2006/122081 (Abuelyaman et al.); U.S. Pat. Nos. 6,495,643 (Evans et al.); 6,344,556 (Evans et al.); 6,043,361 (Evans et al.); WO 96/19471 (Evans et al.); WO 94/14792 (Rizzardo et al.); Richard Evans et al., New Free-Radical Ring-Opening Acrylate Monomers, Macromolecules, 1994, 27 (26), 7935-7937; Richard Evans and Ezio Rizzardo, Free-Radical Ring-Opening Polymerization of Cyclic Allylic Sulfides, Marcomolecules, 1996, 29, 6983-6989; Richard Evans and Ezio Rizzardo, Free Radical Ring-Opening Polymerization of Cyclic Allylic Sulfides. 2. Effect of Substituents on Seven- and Eight-Membered Ring Low Shrink Monomers, Macromolecules, 2000, 33, 6722-6731, and Richard Evans and Ezio Rizzardo, Free Radical Ring-Opening Polymerization of Cyclic Allylic Sulfides: Liquid Monomers with Low Polymerization Volume Shrinkage, Journal of Polymer Science: Part A: Polymer Chemistry, 2001, 39, 202-215.

Vinyl ether compounds that are suitable for use as polymerizable resins include, for example, cyclohexane-1,4-dimethanol divinylether, diethylene glycol divinyl ether, triethylene glycol divinyl ether, and tris(4-(vinyloxy)butyl) trimellitate.

### Initiator System

In certain embodiments, the compositions of the present invention are photopolymerizable, i.e., the compositions contain a polymerizable resin and a photoinitiator system that upon irradiation with actinic radiation initiates the polymerization (or hardening) of the composition. Such photopolymerizable compositions can be free radically polymerizable or cationically polymerizable.

Suitable photoinitiators (i.e., photoinitiator systems that include one or more compounds) for polymerizing free radically polymerizable compositions include binary and tertiary systems. Typical tertiary photoinitiators include an iodonium salt, a photosensitizer, and an electron donor compound as described in U.S. Pat. No. 5,545,676 (Palazzotto et al.). Suitable iodonium salts are the diaryl iodonium salts, e.g., diphenyliodonium chloride, diphenyliodonium hexafluorophosphate, diphenyliodonium tetrafluoroborate, and tolylcumyliodonium tetrakis(pentafluorophenyl)borate. The iodonium salt may be present at about 0.05 to 10.0 weight percent, preferably about 0.20 to 5.0 weight percent, more preferably about 0.40 to 3.0 weight percent, based upon the total weight of the unfilled composition. Suitable photosensitizers are monoketones and diketones that absorb some light within a range of 400 nm to 520 nm (preferably, 450 nm to 500 nm). Particularly suitable compounds include alpha diketones that have light absorption within a range of 400 nm to 520 nm (even more preferably, 450 to 500 nm). Suitable compounds are camphorquinone, benzil, furil, 3,3,6,6-tetramethylcyclohexanedione, phenanthraquinone, 1-phenyl-1,2-propanedione and other 1-aryl-2-alkyl-1,2-ethanediones, and cyclic alpha diketones. The photosensitizer may be present at about 0.05 to 5.0 percent, preferably about 0.10 to 2.0 weight percent, based upon the total weight of the unfilled composition. Suitable electron donor compounds include substituted amines, e.g., ethyl dimethylaminobenzoate. The electron donor compound may be present at about 0.01 to 5.0 weight percent, preferably about 0.05 to 1.0 weight percent, more preferably 0.05 to 0.50 weight percent based upon the total weight of the unfilled composition. Other suitable tertiary photoinitiator systems useful for photopolymerizing cationically polymerizable resins are described, for example, in U.S. Pat. No. 6,765,036 (Dede et al.).

Other useful photoinitiators for polymerizing free radically polymerizable compositions include the class of phosphine oxides that typically have a functional wavelength range of 380 nm to 1200 nm. Preferred phosphine oxide free radical initiators with a functional wavelength range of 380 nm to 450 nm are acyl and bisacyl phosphine oxides such as those described in U.S. Pat. Nos. 4,298,738 (Lechtken et al.), 4,324,744 (Lechtken et al.), 4,385,109 (Lechtken et al.), 4,710,523 (Lechtken et al.), 4,737,593 (Ellrich et al.), and 6,251,963 (Kohler et al.); and EP Application No. 0 173 567 A2 (Ying).

Commercially available phosphine oxide photoinitiators capable of free-radical initiation when irradiated at wavelength ranges of greater than 380 nm to 450 nm include bis(2,4,6-trimethylbenzoyl)phenyl phosphine oxide (IRGACURE 819, Ciba Specialty Chemicals, Tarrytown, N.Y.), bis(2,6-dimethoxybenzoyl)-(2,4,4-trimethylpentyl) phosphine oxide (CGI 403, Ciba Specialty Chemicals), a 25:75 mixture, by weight, of bis(2,6-dimethoxybenzoyl)-2,4,4-trimethylpentyl phosphine oxide and 2-hydroxy-2-methyl-1-phenylpropan-1-one (IRGACURE 1700, Ciba Specialty Chemicals), a 1:1 mixture, by weight, of bis(2,4,6-trimethylbenzoyl)phenyl phosphine oxide and 2-hydroxy-2-methyl-1-phenylpropane-1-one (DAROCUR 4265, Ciba Specialty Chemicals), and ethyl 2,4,6-trimethylbenzylphenyl phosphinate (LUCIRIN LR8893X, BASF Corp., Charlotte, N.C.).

The phosphine oxide initiator may be present in the photopolymerizable composition in catalytically effective amounts, such as from 0.1 weight percent to 5.0 weight percent, based on the total weight of the unfilled composition.

Tertiary amine reducing agents may be used in combination with an acylphosphine oxide. Illustrative tertiary amines useful in the invention include ethyl 4-(N,N-dimethylamino)benzoate and N,N-dimethylaminoethyl methacrylate. When present, the amine reducing agent is present in the photopolymerizable composition in an amount from 0.1 weight percent to 5.0 weight percent, based on the total weight of the unfilled composition. Useful amounts of other initiators are well known to those of skill in the art.

In certain embodiments, the compositions of the present invention are chemically hardenable, i.e., the compositions contain a chemically hardenable component and a chemical initiator (i.e., initiator system) that can polymerize, cure, or otherwise harden the composition without dependence on irradiation with actinic radiation. The chemically hardenable compositions may include redox cure systems that include a polymerizable resin (e.g., an ethylenically unsaturated compound) and redox agents that include an oxidizing agent and a reducing agent. Suitable polymerizable resins, redox agents, optional acid-functional compounds, and optional fillers that are useful in the present invention are described in U.S. Pat. Publication Nos. 2003/0166740 (Mitra et al.) and 2003/0195273 (Mitra et al.).

The reducing and oxidizing agents should react with or otherwise cooperate with one another to produce free-radicals capable of initiating polymerization of the resin system (e.g., the ethylenically unsaturated compound). This type of cure is a dark reaction, that is, it is not dependent on the presence of light and can proceed in the absence of light. The reducing and oxidizing agents are preferably sufficiently shelf-stable and free of undesirable colorization to permit their storage and use under typical dental conditions. They should be sufficiently miscible with the resin system (and preferably water-soluble) to permit ready dissolution in (and discourage separation from) the other components of the composition.

Useful reducing agents include ascorbic acid, ascorbic acid derivatives, and metal complexed ascorbic acid compounds as described in U.S. Pat. No. 5,501,727 (Wang et al.); amines, especially tertiary amines, such as 4-tent-butyl dimethylaniline; aromatic sulfinic salts, such as p-toluenesulfinic salts and benzenesulfinic salts; thioureas, such as 1-ethyl-2-thiourea, tetraethyl thiourea, tetramethyl thiourea, 1,1-dibutyl thiourea, and 1,3-dibutyl thiourea; and mixtures thereof. Other secondary reducing agents may include cobalt (II) chloride, ferrous chloride, ferrous sulfate, hydrazine, hydroxylamine (depending on the choice of oxidizing agent), salts of a dithionite or sulfite anion, and mixtures thereof. Preferably, the reducing agent is an amine.

Suitable oxidizing agents will also be familiar to those skilled in the art, and include but are not limited to persulfuric acid and salts thereof, such as sodium, potassium, ammonium, cesium, and alkyl ammonium salts. Additional oxidizing agents include peroxides such as benzoyl peroxides, hydroperoxides such as cumyl hydroperoxide, t-butyl hydroperoxide, and amyl hydroperoxide, as well as salts of transition metals such as cobalt (III) chloride and ferric chloride, cerium (IV) sulfate, perboric acid and salts thereof, permanganic acid and salts thereof, perphosphoric acid and salts thereof, and mixtures thereof.

It may be desirable to use more than one oxidizing agent or more than one reducing agent. Small quantities of transition metal compounds may also be added to accelerate the rate of redox cure. In some embodiments it may be preferred to include a secondary ionic salt to enhance the stability of the polymerizable composition as described in U.S. Pat. Publication No. 2003/0195273 (Mitra et al.).

The reducing and oxidizing agents are present in amounts sufficient to permit an adequate free-radical reaction rate. This can be evaluated by combining all of the ingredients of the composition except for the optional filler, and observing whether or not a hardened mass is obtained.

Typically, the reducing agent, if used at all, is present in an amount of at least 0.01% by weight, and more typically at least 0.1% by weight, based on the total weight (including water if present) of the components of the composition. Typically, the reducing agent is present in an amount of no greater than 10% by weight, and more typically no greater than 5% by weight, based on the total weight (including water if present) of the components of the composition.

Typically, the oxidizing agent, if used at all, is present in an amount of at least 0.01% by weight, and more typically at least 0.10% by weight, based on the total weight (including water if present) of the components of the composition. Typically, the oxidizing agent is present in an amount of no greater than 10% by weight, and more typically no greater than 5% by weight, based on the total weight (including water if present) of the components of the composition.

The reducing or oxidizing agents can be microencapsulated as described in U.S. Pat. No. 5,154,762 (Mitra et al.). This will generally enhance shelf stability of the composition, and if necessary permit packaging the reducing and oxidizing agents together. For example, through appropriate selection of an encapsulant, the oxidizing and reducing agents can be combined with an acid-functional component and optional filler and kept in a storage-stable state. Likewise, through appropriate selection of a water-insoluble encapsulant, the reducing and oxidizing agents can be combined with an FAS glass and water and maintained in a storage-stable state.

A redox cure system can be combined with other cure systems, including photoinitiator systems or with a composition such as described U.S. Pat. No. 5,154,762 (Mitra et al.).

### Fillers

The hardenable dental compositions of the invention may optionally include at least one filler. For certain embodiments, the at least one filler includes one or more silane-treated nanofillers, selected from nano silica, nano zirconia, zirconia-silica nanoclusters, and combinations thereof.

Suitable fillers may be selected from one or more of a wide variety of materials suitable for incorporation in compositions used for dental applications, such as fillers currently used in dental restorative compositions, and the like.

As indicated above, the filler can affect certain properties of the hardenable dental composition. Appearance is affected in part by adjustment of the amounts and relative refractive indices of the ingredients of the composition, thereby allowing alteration of the translucence, opacity or pearlescence of the composition. In this way, the appearance of the hardenable dental composition after hardening, can, if desired, be made to closely approximate the appearance of natural dentition.

Radiopacity is a measurement of the ability of the dental composite to be detected by x-ray examination. Frequently a radiopaque dental composite will be desirable, for instance, to enable the dentist to determine whether or not a dental restoration remains sound. Under other circumstances a non-radiopaque composite may be desirable. Suitable fillers for radiopaque formulations are described in EP-A2-0 189 540, EP-B-0 238 025, and U.S. Pat. No. 6,306,926 B1 (Bretscher et al.).

The amount of filler that is incorporated into the hardenable dental composition, referred to herein as the “loading level” and expressed as a weight percent based on the total weight of the composition, will vary depending on the type of filler, the resin system and other components of the composition, and the end use of the composition.

For some dental materials, such as sealants, the compositions of the invention can be lightly filled (e.g., having a loading level of less than about 40 weight percent) or unfilled. In such implementations, the viscosity of the composition is sufficiently low to allow its penetration into pits and fissures of occlusal tooth surfaces as well as into etched areas of enamel, thereby aiding in the retention of the dental material. In applications where high strength or durability are desired (e.g., anterior or posterior restoratives, prostheses, crown and bridge cements, artificial crowns, artificial teeth and dentures) the loading level can be as high as about 95 weight percent. For most dental restorative and prosthetic applications a loading level is generally at least 40 weight percent, and more typically is between about 60 and 90 weight percent.

The filler(s) used in the compositions of the invention is typically finely divided. The filler(s) can have a unimodal or polymodal (e.g., bimodal) particle size distribution. The maximum particle size (the largest dimension of a particle, generally, the diameter) of the filler(s) is typically less than 20 micrometers, more typically less than 10 micrometers, and most typically less than 5 micrometers. The average particle size of the filler(s) is typically less than 0.1 micrometers, and more typically less than 0.075 micrometer.

The filler(s) may be an inorganic material. It may also be a crosslinked organic material that is insoluble in the resin system, and is optionally filled with inorganic filler. The filler(s) should in any event be nontoxic and suitable for use in the mouth. The filler(s) can be radiopaque or radiolucent. The filler typically is substantially insoluble in water.

Examples of suitable inorganic fillers are naturally occurring or synthetic materials including, but not limited to: quartz (i.e. silica, SiO₂); nitrides (e.g., silicon nitride); glasses derived from, for example, Zr, Sr, Ce, Sb, Sn, Ba, Zn, and Al; feldspar; borosilicate glass; kaolin; talc; titania; low Mohs hardness fillers such as those described in U.S. Pat. No. 4,695,251 (Randklev); and submicron silica particles (e.g., pyrogenic silicas such as those available under the trade designations AEROSIL, including “OX 50,” “130,” “150” and “200” silicas from Degussa Corp., Akron, Ohio and CAB-O-SIL M5 silica from Cabot Corp., Tuscola, Ill.). In some embodiments, the silica or nanosilica particles are non-pyrogenic, i.e. comprise non-fumed silica. Examples of suitable organic filler particles include filled or unfilled pulverized polycarbonates, polyepoxides, and the like.

The filler may be acid-reactive, non-acid-reactive, or a combination thereof. Suitable non-acid-reactive filler particles include quartz, submicron silica, nanosilica, nanozirconia, and non-vitreous microparticles of the type described in U.S. Pat. No. 4,503,169 (Randklev). Mixtures of these non-acid-reactive fillers are also contemplated, as well as combination fillers made from organic and inorganic materials. Silane-treated zirconia-silica (Zr—Si) filler is especially useful in certain embodiments. In some implementations of the invention, the filler system may contain a combination of at least one filler comprising heavy metal oxide nanoparticles (e.g., zirconia nanoparticles), and/or at least one filler comprising non-heavy metal oxide particles (e.g. silica nanoparticles), and/or at least one filler comprising a heavy metal oxide and a non-heavy metal oxide (e.g. clusters of zirconia and silica nanoparticles (aggregate of nanoziconia and nanosilica)).

Metallic fillers may also be incorporated, such as particulate metal filler made from a pure metal such as those of Groups IVA, VA, VIA, VIIA, VIII, IB, or IIB, aluminum, indium, and thallium of Group IIIB, and tin and lead of Group IVB, or alloys thereof. Conventional dental amalgam alloy powders, typically mixtures of silver, tin, copper, and zinc, may also optionally be incorporated. The particulate metallic filler preferably has an average particle size of about 1 micron to about 100 microns, more preferably 1 micron to about 50 microns. Mixtures of these fillers are also contemplated, as well as combination fillers made from organic and inorganic materials. Fluoroaluminosilicate glass fillers, either untreated or silanol treated, are particularly preferred. These glass fillers have the added benefit of releasing fluoride at the site of dental work when placed in the oral environment.

In some embodiments, the composition may include acid-reactive filler. Suitable acid-reactive fillers include metal oxides, glasses, and metal salts. Typical metal oxides include barium oxide, calcium oxide, magnesium oxide, and zinc oxide. Typical glasses include borate glasses, phosphate glasses, and fluoroaluminosilicate (“FAS”) glasses. In certain embodiments, FAS glasses are particularly preferred. The FAS glass, if present, typically contains sufficient elutable cations so that a hardened dental composition will form when the glass is mixed with the components of the hardenable composition. The glass also typically contains sufficient elutable fluoride ions so that the hardened composition will have cariostatic properties. Such glass can be made from a melt containing fluoride, alumina, and other glass-forming ingredients using techniques familiar to those skilled in the FAS glassmaking art. The FAS glass, if present, is typically in the form of particles that are sufficiently finely divided so that they can conveniently be mixed with the other cement components and will perform well when the resulting mixture is used in the mouth.

Generally, the average particle size (typically, diameter) for FAS glass used in such compositions is no greater than about 12 micrometers, typically no greater than 10 micrometers, and more typically no greater than 5 micrometers as measured using, for example, a sedimentation analyzer. Suitable FAS glasses will be familiar to those skilled in the art, and are available from a wide variety of commercial sources, and many are found in currently available glass ionomer cements such as those commercially available under the trade designations VITREMER, VITREBOND, RELY X LUTING CEMENT, RELY X LUTING PLUS CEMENT, PHOTAC-FIL QUICK, KETAC-MOLAR, and KETAC-FIL PLUS (3M ESPE Dental Products, St. Paul, Minn.), FUJI II LC and FUJI IX (G-C Dental Industrial Corp., Tokyo, Japan) and CHEMFIL Superior (Dentsply International, York, Pa.). Mixtures of fillers can be used if desired.

The surface of the filler particles can also be treated with a coupling agent in order to enhance the bond between the filler and the resin. Suitable coupling agents include gamma-methacryloxypropyltrimethoxysilane, gamma-mercaptopropyltriethoxysilane, gamma-aminopropyltrimethoxysilane, and the like. Silane-treated zirconia-silica (ZrO₂—SiO₂) fillers and nanofillers, silane-treated silica fillers and nanofillers, silane-treated zirconia fillers and nanofillers, and combinations thereof are especially suitable for certain restorative compositions.

Other suitable fillers are disclosed in U.S. Pat. Nos. 6,387,981 (Zhang et al.); 6,572,693 (Wu et al.); 6,730,156 (Windisch); and 6,899,948 (Zhang); as well as in International Publication No. WO 03/063804 (Wu et al.). Filler components described in these references include nanosized silica particles, nanosized metal oxide particles, and combinations thereof. Nanofillers are also described in U.S. Patent Publication Nos. 2005/0252413 (Kangas et al.); 2005/0252414 (Craig et al.); and 2005/0256223 (Kolb et al.).

For some embodiments of the present invention that include filler (e.g., dental adhesive compositions), the compositions typically include at least 1% by weight, more typically at least 2% by weight, and most typically at least 5% by weight filler, based on the total weight of the composition. For such embodiments, compositions of the present invention typically include at most 40% by weight, more typically at most 20% by weight, and most typically at most 15% by weight filler, based on the total weight of the composition.

For other embodiments (e.g., wherein the composition is a dental restorative or an orthodontic adhesive), compositions of the present invention typically include at least 40% by weight, more typically at least 45% by weight, and most typically at least 50% by weight filler, based on the total weight of the composition. For such embodiments, compositions of the present invention typically include at most 90% by weight, more typically at most 80% by weight, even more typically at most 70% by weight filler, and most typically at most 50% by weight filler, based on the total weight of the composition.

### Other Additives

Optionally, compositions of the present invention may contain solvents (e.g., alcohols (e.g., propanol, ethanol), ketones (e.g., acetone, methyl ethyl ketone), esters (e.g., ethyl acetate), other nonaqueous solvents (e.g., dimethylformamide, dimethylacetamide, dimethylsulfoxide, 1-methyl-2-pyrrolidinone)), or mixtures thereof.

In some embodiments, the compositions are non-aqueous. In other embodiments, the compositions may optionally contain water. The water can be distilled, deionized, or plain tap water. If present, the amount of water should be sufficient to provide adequate handling and mixing properties and/or to permit the transport of ions, particularly in a filler-acid reaction. In such embodiments, water represents at least about 1 weight percent, and more preferably at least about 5 weight percent, of the total weight of components used to form the hardenable composition. Generally, water represents no greater than about 75 weight percent, and more preferably no greater than about 50 weight percent, of the total weight of components used to form the hardenable composition.

If desired, the compositions of the invention may contain additives such as indicators, dyes (including photobleachable dyes), pigments, inhibitors, accelerators, viscosity modifiers, wetting agents, antioxidants, tartaric acid, chelating agents, buffering agents, stabilizers, diluents, and other similar ingredients that will be apparent to those skilled in the art. Surfactants, for example, nonionic surfactants, cationic surfactants, anionic surfactants, and combinations thereof, may optionally be used in the compositions. Useful surfactants include non-polymerizable and polymerizable surfactants. Additionally, medicaments or other therapeutic substances can be optionally added to the dental compositions. Examples include, but are not limited to, fluoride sources, whitening agents, anticaries agents (e.g., xylitol), remineralizing agents (e.g., calcium phosphate compounds and other calcium sources and phosphate sources), enzymes, breath fresheners, anesthetics, clotting agents, acid neutralizers, chemotherapeutic agents, immune response modifiers, thixotropes, polyols, anti-inflammatory agents, antimicrobial agents, antifungal agents, agents for treating xerostomia, desensitizers, and the like, of the type often used in dental compositions. Combinations of any of the above additives may also be employed. The type and amount of any one such additive can be selected by one of skill in the art to accomplish the desired result without undue experimentation.

### Preparation of Compounds of Formula I

2,5-Dialkoxyterephthalates can be prepared by alkylating 2,5-dihydroxyterephthalates using conventional methods. For example, a solution of the dihydroxyterephthalate can be treated with a dialkyl sulfate, such as dimethyl sulfate, in the presence of a base. The reaction can be conveniently carried out in a suitable solvent such as acetone and at an elevated temperature, such as the reflux temperature of the solvent. Alternatively, the alkylation reaction can be carried out using a Williamson-type ether synthesis. The reaction can be conveniently carried out by combining an alkyl halide with the 2,5-dihydroxyphthalate in a solvent such as DMF in the presence of a suitable base such as cesium carbonate. The reaction can be carried out at ambient temperature or at an elevated temperature, for example, 65° C. or 85° C. The alkyl halide may be substituted, for example, by (meth)acryloyloxy. In this case, one or more polymerizable groups may be included in the compound of Formula I.

Compounds where R₁ and R₂ are alkenyl, alkynyl, phenyl, heteroaryl, and/or heterocyclyl can be made by known methods. For example, an alkenyl group may be provided by displacing the acetate group from an alkenyl acetate, for example, vinyl acetate, with a phenol (e.g., Adelman et al., J. Am. Chem. Soc.; v. 75; 1953; pg. 2678), an alkynyl group may be provided by reacting an alkynyl phenyl iodonium triflate with a phenoxide anion (e.g., Nikas et al., Molecules; v. 5; 2000; pg. 1182-1186), a phenyl group may be provided using an Ullmann coupling reaction, a heteroaryl group may be provided by treating a phenol in a basic solution with a halogen substituted heteroaryl, for example, 2-bromo pyridine (e.g., den Hertog; de Jonge; Recl. Tray. Chim. Pays-Bas; v. 67; 1948; pg. 385-391), and a heterocyclyl group may be provided by treating a phenol in a basic solution with a halogen substituted heterocyclyl, for example, 3-chloro-1-methylpiperidine (e.g., U.S. Pat. No. 2,831,862 (Biel))

### Preparation and Use of the Compositions

The hardenable dental compositions of the present invention can be prepared by combining all the various components using conventional mixing techniques. The resulting composition may optionally contain fillers, solvents, water, and other additives as described herein. Typically, photopolymerizable compositions of the invention are prepared by simply admixing, under “safe light” conditions, the components of the inventive compositions. Suitable inert solvents may be employed if desired when effecting this mixture. Any solvent may be used which does not react appreciably with the components of the inventive compositions. Examples of suitable solvents include acetone, methyl ethyl ketone, ethyl acetate, isopropanol, and ethanol. A liquid material to be polymerized may be used as a solvent for another liquid or solid material to be polymerized. Solventless compositions can be prepared by simply dissolving any initiator system (e.g., an iodonium complex salt, sensitizer, and electron donor) in the polymerizable resin, with or without the use of mild heating to facilitate dissolution.

The amounts and types of each component in the dental material should be adjusted to provide the desired physical and handling properties before and after polymerization. For example, the polymerization rate, polymerization stability, fluidity, compressive strength, tensile strength and durability of the dental material typically are adjusted in part by altering the types and amounts of polymerization initiator(s) and, if present, the loading and particle size distribution of filler(s). Such adjustments typically are carried out empirically based on previous experience with dental materials. When the dental material is applied to a tooth, the tooth can optionally be pre-treated with a primer and/or an adhesive by methods known to those skilled in the art.

The compositions can be supplied in a variety of forms including one-part systems and multi-part systems, e.g., two-part powder/liquid, paste/liquid, paste/powder and paste/paste systems. Other forms employing multi-part combinations (i.e., combinations of two or more parts), each of which is in the form of a powder, liquid, gel, or paste are also possible. The various components of the composition may be divided up into separate parts in whatever manner is desired; however, in a redox multi-part system, one part typically contains the oxidizing agent and another part typically contains the reducing agent, though it is possible to combine the reducing agent and oxidizing agent in the same part of the system if the components are kept separated, for example, through use of microencapsulation. Also, for those embodiments in which the dental composition is a resin-modified glass ionomer (RMGI), the polyacid, acid-reactive filler, and water generally would not all be present in the same part, although any two of these may be grouped together in the same part along with any combination of other components.

The components of the composition can be included in a kit, where the contents of the composition are packaged to allow for storage of the components until they are needed.

The components of the composition can be mixed and clinically applied using conventional techniques. A curing light is generally required for the initiation of photopolymerizable compositions. The compositions may be in the form of composites or restoratives that adhere very well to dentin and/or enamel. Optionally, a primer layer can be used on the tooth tissue on which the hardenable composition is used.

The invention encompasses a wide variety of dental compositions, which may be filled or unfilled. Exemplary dental materials include dental restoratives (e.g., composites, fillings, sealants, inlays, onlays, crowns, and bridges), orthodontic appliances, and orthodontic adhesives. Such dental materials include direct aesthetic restorative materials (e.g., anterior and posterior restoratives), prostheses, adhesives and primers for oral hard tissues, sealants, veneers, cavity liners, orthodontic bracket adhesives for use with any type of bracket (such as metal, plastic and ceramic), crown and bridge cements, artificial crowns, artificial teeth, dentures, and the like. These dental materials are used in the mouth and are disposed adjacent to natural teeth. The phrase “disposed adjacent to” as used herein refers to the placing of a dental material in temporary or permanent bonding (e.g., adhesive) or touching (e.g., occlusal or proximal) contact with a natural tooth.

The features and advantages of this invention are further illustrated by the following examples, which are in no way intended to be limiting thereof. The particular materials and amounts thereof recited in these examples, as well as other conditions and details, should not be construed to unduly limit this invention. Unless otherwise indicated, all parts and percentages are on a weight basis, all water is deionized water, and all molecular weights are weight average molecular weight.

### EXAMPLES

Unless otherwise noted, reagents and solvents were obtained from Sigma-Aldrich Corp., St. Louis, Mo.

### Abbreviations, Descriptions, and Sources of Materials

| Abbreviation | Description and Source of Material |
|---|---|
| BisGMA | 2,2-bis[4-(2-hydroxy-3- |
| | methacryloyloxypropoxy)phenyl]propane CAS No. 1565-94-2 |
| TEGDMA | triethyleneglycol dimethacrylate (Sartomer Co., Inc., Exton, PA)) |
| UDMA | diurethane dimethacrylate (ROHAMERE 6661-0 from Rohm |
| | America LLC, Piscataway, NJ) |
| BisEMA6 | ethoxylated bisphenol A dimethacrylate (Sartomer Co., Inc., Exton, PA) |
| ENMAP | ethyl N-methyl-N-phenyl-3-aminopropionate, prepared by |
| | Michael addition of N-methylaniline to ethyl acrylate (J. |
| | Chem. Soc., Supplement, p. 144-152 (1949)) |
| CAMP | 1-(2-methacryloyloxyethyl)-2-(7-methylene-1,5-dithiaoctan-3 |
| | yl) phlatate, prepared as described in Example 1 of PCT |
| | International Publication No. US2006/122081 |
| TrisMAP | 2-[4-(2-hydroxy-3-methacryloxypropoxy)phenyl]-2-[4-(2-[[2- |
| | methacryloxyethoxy]phthalyloxy]-3- |
| | methacryloxypropoxy)phenyl]-propane, prepared as described below |
| CPQ | camphorquinone |
| BHT | 2,6-di-tert-butyl-4-methylphenol |
| EDMAB | ethyl 4-(N,N-dimethylamino)benzoate |
| DPIHFP | diphenyl iodonium hexafluorophosphate (Johnson Matthey, |
| (DPIPF₆) | Alpha Aesar Division, Ward Hill, NJ) |
| TINUVIN | refers to a polymerizable UV stabilizer obtained under the |
| | trade designation TINUVIN R 796 (Ciba Specialty Chemicals, Tarrytown, NY) |
| IRGACURE | bis(2,4,6-trimethylbenzoyl)phenyl phosphine oxide (Ciba |
| 819 | Specialty Chemicals, Tarrytown, NY) |
| Zr—Si Filler | silane-treated zirconia-silica nanocluster filler, prepared |
| | essentially as described in U.S. Pat. No. 6,730,156 |
| Silica Filler | silane-treated nano-sized silica having a nominal particle size |
| | of approximately 20 nanometers, prepared essentially as |
| | described for FILLER F in U.S. Patent Publication No. 2005/0252413 |
| Zirconia Filler | silane-treated nano-sized zirconia, prepared essentially as |
| | described in Preparatory Example 1A in U.S. Patent |
| | Publication No. 2005/0252413 |
| LUMILUX | diethyl 2,5-dihydroxyterephthalate (Honeywell Inc., |
| BLUE LZ | Morristown, NJ) |
| LUMILUX | inorganic fluorescent pigment (Honeywell Inc., Morristown, |
| WHITE | NJ) |
| Red Pigment | a dispersion containing a red iron III oxide pigment |
| Dispersion | |
| Yellow Pigment | a dispersion containing a yellow iron III oxide pigment |
| Dispersion | |
| White Pigment | a dispersion containing a rutile titanium dioxide pigment |
| Dispersion | |

### Preparation of Diethyl 2,5-Dimethoxyterephthalate

Diethyl 2,5-hydroxyterephthalate (1.00 g) and anhydrous potassium carbonate (2.02 g) were suspended in acetone (25 ml) in a 100 ml round bottom flask. Dimethyl sulfate (2.01 g) in acetone (8 ml) was added dropwise to the resulting yellow suspension, using an addition funnel fitted with a moisture guard tube containing DRIERITE. The reaction mixture was held at room temperature with constant mixing during the addition. No exotherm was noted during the addition. After addition was complete (10 min), the addition funnel was replaced with a condenser, and the resulting solution was refluxed for 4 hours. Thin layer chromatography using hexanes:ethylacetate (80:20) indicated that starting material was still present. An additional 0.5889 g of dimethyl sulfate was added all at once, and the resulting mixture was refluxed for another 6 hours.

The acetone was removed from the mixture under rotary evaporation to yield a colorless solid. Water was added to the round bottom flask, and the solid was extracted using methyl tent-butyl ether (MTBE, 3×50 ml). The MTBE extracts were combined and washed several times with 10% aqueous sodium hydroxide solution and then washed with a pH 7 buffer (available as stock number 38712 from Alfa-Aesar, a Johnson Mattley Company, Lancashire, England) until the washings tested neutral on pH paper. The resulting washed MTBE extract was dried over anhydrous magnesium sulfate and then evaporated to dryness using a rotary evaporator to provide a colorless solid (0.9801 g). NMR (proton and carbon-13) revealed that this solid was the desired product, diethyl 2,5-dimethoxyterephthalate. ¹H NMR data in CDCl₃: δ ppm (multiplicity, H) 1.40 (triplet, 6H), 3.89 (singlet, 6H), 4.39 (quartet, 4H), 7.38 (singlet, 2H).

### Preparation of TrisMAP

A 3-neck flask, fitted with a mechanical stirrer, an addition funnel, and a source of nitrogen gas, was charged with bisGMA (342.5 g), mono-2-(methacryloyloxyethyl) phthalate (181 g), 4-(dimethylamino)pyridine (6.4 g), and ethyl acetate (400 mL). The mixture was mechanically stirred while the flask was cooled in a bath of ice. After approximately 20 minutes, a solution of dicyclohexylcarbodiimide (136 g) in ethyl acetate (150 mL) was added dropwise to the cold stirring mixture. The cold mixture was then stirred for approximately one hour, after which time it was allowed to warm to room temperature. The mixture was then stirred overnight at room temperature. The mixture was then vacuum filtered and the filtrate was washed with 1N aqueous HCl (200 mL). The organic phase was dried over anhydrous sodium sulfate and was concentrated under vacuum. The resultant viscous liquid was vacuum filtered using a fitted glass funnel to afford the product as a clear colorless liquid.

### Test Methods

### Fluorescence CIE 2° and Intensity Test Method

The fluorescence of disk samples described in Examples 1-5 and Comparative Examples 1-2 was measured using a Tecan Infinite 200 fluorescence spectrometer (Tecan US Ltd., Durham, N.C.). All samples were irradiated using an excitation wavelength of 365 nm, and emission wavelengths between 390 nm and 730 nm were monitored at 1 nm intervals. The gain was set manually at 70, and each data point was integrated for 20 microseconds with 25 readings per data point. The resulting data was analyzed by known methods as described in Section 3.3.8 in Gunter Wyszecki and W. S. Stiles, “Color Science. Concepts and Methods. Quantitative Data and Formulae.”, 2nd edition, John Wiley (1982) and/or in Gunter Wyszecki and W. S. Stiles, Color Science: Concepts and Methods, Quantitative Data and Formulae”, 2nd edition, Wiley-Interscience (2000). Data for the 1931 CIE 2° standard observer color matching functions are known and were obtained from the CIE publication, CIE 15:2004 (ISBN 3901906339). The results of the analysis were reported in terms of CIE 2° Chromaticity Coordinates, x and y, which correspond to the perceived color of fluorescence.

Fluorescence intensity was determined from the above collected data by integrating the emission spectrum and reported as counts per second (cps). Fluorescence intensity values of about 500,000 cps are typical for natural tooth fluorescence, although, as indicated above, natural tooth fluorescence can vary significantly between subjects. Since fluorescence intensity is highly dependent upon the particular instrument used and the configuration and dimensions of the sample, any comparative testing normally involves using the same instrument and using samples with the same dimensions and shape.

### Color Test Method

Disk samples prepared as described in the Examples and Comparative Examples were evaluated for color using the known CIELAB measurement system. CIELAB color data was obtained on the disks using a Hunterlab UltraScan VIS spectrophotometer (Hunter Associates Laboratory, Inc., Reston, Va.). Color coordinates, L*, a*, and b*, and the Contrast Ratio were obtained on each disk sample. The b* coordinate is a measure of yellowness, a higher b* value indicating more yellowness. The contrast ratio is an indication of translucency, a lower contrast ratio indicating a greater degree of translucency.

### Examples 1-5

Hardenable dental compositions containing polymerizable resin mixtures were prepared by combining the components listed in Tables 1 to 5 in a Model DAC 150 FVZ SpeedMixer (manufactured by FlackTek, Inc., Landrum, S.C.) using progressively higher speeds from 1500 to 3500 rpm in separate one-minute mixing cycles with cooling between mix cycles until no further change in the resulting mixture was observed. The weight percentages given in Tables 1 to 5 are the weight percentages of the components in the final compositions (composites). All polymerizable resin mixture components were first combined and then added to silica filler, Zr—Si filler, and any pigments, to provide a hardenable dental composition containing the amount of each component shown in respective Tables 1 to 5. Each composition was loaded into a syringe and de-bubbled by applying a load to the syringe plunger for 16 hours at 45° C. The compositions were stored and handled under yellow lights (“safe lights”). A disk of each composition having a thickness of 1.1 millimeter and a diameter of 30 millimeters was prepared by pressing the composition in a stainless steel mold in a hydraulic press (Model 3912 obtained from Carver, Inc., Wabash, Ind.) at 68.95 MPa (10,000 pounds per square inch) pressure for 90 seconds. The press had been fitted with fiber optic cables to direct light to the composition from a Model A20500 ACE light source (Schott North America, Inc., Auburn, N.Y.). The composition was then cured (photopolymerized) for two minutes while still under pressure. The resulting disk was removed from the press and was further cured for 90 seconds using a high intensity pulsed Xenon light source (UniXS, Heraeus Kulzer, Inc., Armonk, N.Y.). Fluorescence of the resulting hardened disk was determined as described above under Fluorescence CIE 2° Test Method. The resulting values for the Chromaticity Coordinates x and y are shown in Table 8. For comparison, uncut and unprophyed bovine teeth were potted in polymethylmethacrylate resin, and the fluorescence was determined as for the Examples. For additional comparison, Table 8 also includes fluorescence data determined in the same way for dentin taken from subjects 10, 58, and 77 years old.

TABLE 1
Composition Of Example 1

| | | Weight Percentage in |
|---|---|---|
| Component | Parts By Weight | Composition |
| ENMAP | 0.01530 | 0.19 |
| CPQ | 0.003429 | 0.04 |
| TINUVIN | 0.008996 | 0.11 |
| BHT | 0.002341 | 0.03 |
| UDMA | 0.1728 | 2.15 |
| BisEMA6 | 0.2462 | 3.07 |
| CAMP | 0.3958 | 4.93 |
| TrisMAP | 1.1591 | 14.45 |
| Diethyl 2,5- | 0.001985 | 0.025 |
| dimethoxyterephthalate | | |
| Zr—Si Filler | 5.4428 | 67.84 |
| Silica Filler | 0.5738 | 7.15 |

TABLE 2
Composition Of Example 2

| | | Weight Percentage in |
|---|---|---|
| Component | Parts By Weight | Composition |
| ENMAP | 0.01529 | 0.19 |
| CPQ | 0.003437 | 0.04 |
| TINUVIN | 0.008983 | 0.11 |
| BHT | 0.002416 | 0.03 |
| UDMA | 0.1725 | 2.15 |
| BisEMA6 | 0.2455 | 3.06 |
| CAMP | 0.3948 | 4.93 |
| TrisMAP | 1.1561 | 14.43 |
| Diethyl 2,5- | 0.000996 | 0.013 |
| dimethoxyterephthalate | | |
| LUMILUX WHITE | 0.0095 | 0.12 |
| Zr—Si Filler | 5.4295 | 67.79 |
| Silica Filler | 0.5702 | 7.12 |

TABLE 3
Composition Of Example 3

| | | Weight Percentage in |
|---|---|---|
| Component | Parts By Weight | Composition |
| ENMAP | 0.01913 | 0.19 |
| CPQ | 0.00421 | 0.04 |
| TINUVIN | 0.01116 | 0.11 |
| IRGACURE 819 | 0.01240 | 0.12 |
| UDMA | 0.2143 | 2.14 |
| BisEMA6 | 0.3051 | 3.05 |
| CAMP | 0.4884 | 4.89 |
| TrisMAP | 1.4334 | 14.35 |
| Diethyl 2,5- | 0.000697 | 0.0079 |
| dimethoxyterephthalate | | |
| LUMILUX WHITE | 0.01284 | 0.13 |
| Zr—Si Filler | 6.7604 | 67.66 |
| Silica Filler | 0.7067 | 7.07 |
| Red Pigment | 0.00143 | 0.01 |
| Dispersion | | |
| Yellow Pigment | 0.00735 | 0.07 |
| Dispersion | | |
| White Pigment | 0.01422 | 0.14 |
| Dispersion | | |

TABLE 4
Composition Of Example 4

| | | Weight Percentage in |
|---|---|---|
| Component | Parts By Weight | Composition |
| ENMAP | 0.01677 | 0.19 |
| CPQ | 0.003689 | 0.04 |
| TINUVIN | 0.009776 | 0.11 |
| IRGACURE 819 | 0.01087 | 0.12 |
| UDMA | 0.1878 | 2.14 |
| BisEMA6 | 0.2674 | 3.05 |
| CAMP | 0.4279 | 4.89 |
| TrisMAP | 1.2559 | 14.35 |
| Diethyl 2,5- | 0.000916 | 0.01 |
| dimethoxyterephthalate | | |
| LUMILUX WHITE | 0.01108 | 0.13 |
| Zr—Si Filler | 5.9234 | 67.66 |
| Silica Filler | 0.6191 | 7.07 |
| Red Pigment | 0.00121 | 0.011 |
| Dispersion | | |
| Yellow Pigment | 0.00645 | 0.07 |
| Dispersion | | |
| White Pigment | 0.01243 | 0.14 |
| Dispersion | | |

TABLE 5
Composition Of Example 5

| | | Weight Percentage in |
|---|---|---|
| Component | Parts By Weight | Composition |
| ENMAP | 0.01642 | 0.19 |
| CPQ | 0.003613 | 0.04 |
| TINUVIN | 0.009574 | 0.11 |
| IRGACURE 819 | 0.01064 | 0.12 |
| UDMA | 0.1839 | 2.14 |
| BisEMA6 | 0.2619 | 3.05 |
| CAMP | 0.4191 | 4.89 |
| TrisMAP | 1.2300 | 14.34 |
| Diethyl 2,5- | 0.001111 | 0.013 |
| dimethoxyterephthalate | | |
| LUMILUX WHITE | 0.01085 | 0.13 |
| Zr—Si Filler | 5.8017 | 67.66 |
| Silica Filler | 0.6066 | 7.07 |
| Red Pigment | 0.00116 | 0.011 |
| Dispersion | | |
| Yellow Pigment | 0.00631 | 0.07 |
| Dispersion | | |
| White Pigment | 0.01221 | 0.14 |
| Dispersion | | |

### Comparative Examples 1 and 2

Compositions were prepared as described in Examples 1 to 5, except that no diethyl 2,5-methoxyterephthalate was used. The amount of each component used in these compositions is shown in the following Tables 6 and 7. The compositions were used to make disks as described in Examples 1 to 5.

TABLE 6
Composition Of Comparative Example 1

| | Parts By | Weight Percentage |
|---|---|---|
| Component | Weight | in Composition |
| ENMAP | 0.01428 | 0.19 |
| CPQ | 0.003221 | 0.04 |
| TINUVIN | 0.008387 | 0.11 |
| BHT | 0.00233 | 0.03 |
| UDMA | 0.1609 | 2.14 |
| BisEMA6 | 0.2289 | 3.05 |
| CAMP | 0.3683 | 4.90 |
| TrisMAP | 1.07813 | 14.35 |
| LUMILUX WHITE | 0.0182 | 0.24 |
| Zr—Si Filler | 5.0987 | 67.86 |
| Silica Filler | 0.5319 | 7.08 |

TABLE 7
Composition Of Comparative Example 2

| | Parts By | Weight Percentage |
|---|---|---|
| Component | Weight | in Composition |
| ENMAP | 0.01727 | 0.19 |
| CPQ | 0.0038 | 0.04 |
| TINUVIN | 0.01007 | 0.11 |
| IRGACURE 819 | 0.01119 | 0.12 |
| UDMA | 0.1934 | 2.15 |
| BisEMA6 | 0.2754 | 3.06 |
| CAMP | 0.4408 | 4.89 |
| TrisMAP | 1.2938 | 14.35 |
| LUMILUX WHITE | 0.01123 | 0.12 |
| Zr—Si Filler | 6.09968 | 67.66 |
| Silica Filler | 0.6378 | 7.07 |
| Red Pigment | 0.00129 | 0.01 |
| Dispersion | | |
| Yellow Pigment | 0.00665 | 0.07 |
| Dispersion | | |
| White Pigment | 0.01284 | 0.14 |
| Dispersion | | |

TABLE 8
Fluorescence Intensity For Examples 1-5 And Comparative
Examples 1 And 2; And Chromaticity Coordinate Values
For Examples 1-5, Comparative Examples 1 And 2, Bovine
Enamel, and Literature Values For Dentin.

| | Chromaticity Coordinate | | Fluorescence |
|---|---|---|---|
| | x | y | Intensity (cps) |
| Example 1 | 0.16 | 0.06 | 854818 |
| Example 2 | 0.16 | 0.09 | 653069 |
| Example 3 | 0.16 | 0.09 | 412509 |
| Example 4 | 0.16 | 0.09 | 466677 |
| Example 5 | 0.16 | 0.09 | 551184 |
| Comparative Example 1 | 0.16 | 0.13 | 574726 |
| Comparative Example 2 | 0.17 | 0.15 | 206795 |
| Bovine Enamel Sample 1 | 0.19 | 0.15 | — |
| Bovine Enamel Sample 2 | 0.18 | 0.14 | — |
| Dentin (10 year)¹ | 0.19 | 0.21 | — |
| Dentin (58 year)¹ | 0.18 | 0.19 | — |
| Dentin (77 year)¹ | 0.18 | 0.19 | — |

¹Data for these materials was taken from Matsumoto et al., Arch. Oral. Biol., 44, 309 (1999).

Examples 1-5 provided a natural tooth fluorescence. However, the presence of LUMILUX WHITE made Examples 2-5 visibly more yellow. In addition, the presence of the pigments in Examples 3-5 reduced the fluorescence intensity. Example 5 showed that an increase in the level of the diethyl 2,5-dimethoxyterephthalate easily overcame the reduced fluorescence intensity caused by the pigments. The fluorescence of Example 1 was a natural tooth fluorescence without any yellowing of the composite.

Comparative Example 1 was excessively yellow, because of the large amount of LUMILUX WHITE present in the material. Comparative Example 2 had a very low fluorescence intensity.

### Example 6

A polymerizable resin mixture was prepared by combining the components listed in Table 9 using a Model DAC 150 FVZ SpeedMixer at 3000 rpm in three separate one-minute mixing cycles. The weight percentages given in Table 9 are the weight percentages of the components in the resin mixture. Sufficient diethyl 2,5-dimethoxyterephthalate was then combined with the resin mixture to provide a resin mixture containing 0.465 weight percent diethyl 2,5-dimethoxyterephthalate. To this diethyl 2,5-dimethoxyterephthalate-containing polymerizable resin mixture were then added zirconia filler, silica filler, and Zr—Si filler to provide a hardenable dental composition containing 78.5 weight percent filler mixture, the filler mixture having a composition of 90.5 weight percent Zr—Si filler, and 9.5 weight percent of a mixture of 27 weight percent zirconia filler and 73 weight percent silica filler. This hardenable dental composition contained 0.10 weight percent diethyl 2,5-dimethoxyterephthalate. A disk of this composition was prepared as described in Examples 1-5. The color of the disk sample was measured as described above under Color Test Method. The results are shown in Table 10. In addition, color stability of the disk sample was determined by exposing the disk sample to a medium pressure Xenon arc lamp while the sample was submerged in 37° C. deionized water for a period of 24 hours, and then the color of the disk sample was again measured. The change in color, DE*, was then calculated, and the results are shown in Table 10.

TABLE 9
Composition Of Polymerizable Resin Mixture Of Example 6.

| Component | Weight Percentage in Resin |
|---|---|
| bisGMA | 24.20 |
| UDMA | 33.88 |
| bisEMA6 | 33.88 |
| TEGDMA | 4.84 |
| CPQ | 0.16 |
| DPIHFP | 0.48 |
| EDMAB | 0.97 |
| BHT | 0.15 |
| TINUVIN | 1.45 |

### Comparative Example 3

A hardenable composition was prepared as described in Example 6, except that the diethyl 2,5-dimethoxyterephthalate was replaced by LUMILUX BLUE LZ. A disk sample was prepared from this composition as described in Example 6. Color and color stability were measured as described in Example 6, and the results are shown in Table 10.

TABLE 10
Color Data For Compositions Of Example
6 And Comparative Example 3.

| | Exposure | | | | Contrast | |
|---|---|---|---|---|---|---|
| | Time (hrs) | L* | a* | b* | Ratio | DE*¹ |
| Example 6 | 0 | 87.39 | −3.58 | 11.91 | 43.97 | — |
| Example 6 | 24 | 82.66 | −1.46 | 21.39 | 45.14 | 10.80 |
| Comparative | 0 | 93.96 | −31.13 | 57.26 | 46.1 | — |
| Example 3 | | | | | | |
| Comparative | 24 | 88.71 | −22.03 | 49.02 | 46.87 | 13.35 |
| Example 3 | | | | | | |

¹DE* = [(L*₀ − L*₂₄)² + (a*₀ − a*₂₄)² + (b*₀ − b*₂₄)²]^{0.5}

The results in Table 10 show that Example 6 had a far lower degree of yellowness and a higher translucency, both before and after the 24 hour exposure, than Comparative Example 3. In addition, the color of Example 6 was more stable as indicated by its lower DE* value.

Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein.

The complete disclosures of the patents, patent documents, and publications cited herein are incorporated by reference in their entirety or the portions of each that are indicated as if each were individually incorporated.

1. A hardenable dental composition comprising:
a fluorescent compound of the Formula I: 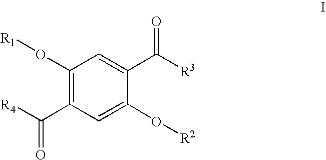
wherein:
R₁ and R₂ are independently selected from the group consisting of alkyl, alkenyl, alkynyl, phenyl, heteroaryl, and heterocyclyl; each of which is unsubstituted or substituted with one or more substituents independently selected from the group consisting of alkyl, alkenyl, alkoxy, haloalkyl, haloalkoxy, halogen, hydroxy, mercapto, cyano, carboxy, alkoxycarbonyl, alkyloyloxy, acryloyloxy, methacryloyloxy, alkoxyalkylene, glycidoxy, formyl, phenyl, phenoxy, phenylalkoxy, phosphono, dialkylphosphono, phosphonooxy, dialkylphosphonooxy, alkylsulfonyl, phenylsulfonyl, heteroaryl, heteroaryloxy, heteroarylalkoxy, heterocyclyl, heterocyclylalkylenyl, and in the case of alkyl, alkenyl, alkynyl, and heterocyclyl, oxo and epoxy;
R₃ and R₄ are independently selected from the group consisting of HO—, alkyl-O—, (meth)acryloyl-O-alkylene-O—, phenyl-O—, heteroaryl-O—, and M⁺⁻O—; wherein M⁺ is a monovalent metal cation; and
a resin system; and
wherein the composition upon hardening has a natural tooth fluorescence.

2. The composition of claim 1, wherein R₁ and R₂ are each independently alkyl, acryloyloxyalkylenyl, methacryloyloxyalkylenyl, glycidoxyalkylenyl, or glycidoxyphenyl.

3. The composition of claim 1, wherein R₃ and R₄ are each independently alkoxy.

4. The composition of claim 3, wherein R₁ and R₂ are methyl and R₃ and R₄ are ethoxy.

5. The composition of claim 1, wherein the fluorescence from the composition upon hardening has chromaticity coordinates x and y, wherein x is in the range of 0.13 to 0.19, and y is in the range of 0.05 to 0.22.

6. The composition of claim 5, wherein x is in the range of 0.14 to 0.18, and y is in the range of 0.09 to 0.20.

7. The composition of claim 1, wherein the fluorescence has a wavelength of maximum emission, wherein the wavelength is in the range of 410 nm to 475 nm.

8. The composition of claim 7, wherein the wavelength is in the range of 420 nm to 460 nm.

9. The composition of claim 1, further comprising at least one filler.

10. The composition of claim 9, wherein the at least one filler includes zirconia.

11. The composition of claim 10, wherein the at least one filler includes an aggregate of nanozirconia and nanosilica.

12. The composition of claim 1, wherein the fluorescent compound of the Formula I is present in an amount of 0.0001 to 0.5 weight percent based upon the weight of the composition.

13. The composition of claim 1, wherein the fluorescent compound of the Formula I is present in an amount of 0.01 to 1.0 weight percent based upon the weight of the resin system.

14. The composition of claim 1, wherein the resin system comprises a polymerizable resin and an initiator system.

15. The composition of claim 14, wherein the polymerizable resin comprises an ethylenically unsaturated compound, an epoxy compound, or a combination thereof.

16. The composition of claim 15, wherein the polymerizable resin comprises an ethylenically unsaturated compound.

17. The composition of claim 15, wherein the ethylenically unsaturated compound is a (meth)acrylate.

18. The composition of claim 15, wherein the polymerizable resin comprises an epoxy compound.

19. The composition of claim 1, further comprising at least one additional fluorescent material having a maximum wavelength of emission in the range of 400 nm to 520 nm.

20. A dental article made by hardening a composition of claim 1.

21. A method of making a dental article, the method comprising the steps of
providing a hardenable dental composition of claim 1; and
hardening the composition.

22. The method of claim 21, wherein hardening the composition is carried out by photopolymerizing the hardenable dental composition.

23. A method of making a hardenable dental composition which upon hardening has natural tooth fluorescence, the method comprising the steps of:
providing a hardenable material that, upon hardening, provides a material that has a non-natural tooth fluorescence; and
adding a sufficient amount of a fluorescent compound of Formula I to the hardenable material such that, upon hardening, the material has a natural tooth fluorescence;
wherein the fluorescent compound of Formula I is: 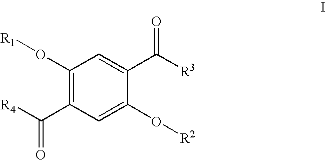
wherein:
R₁ and R₂ are independently selected from the group consisting of alkyl, alkenyl, alkynyl, phenyl, heteroaryl, and heterocyclyl; each of which is unsubstituted or substituted with one or more substituents independently selected from the group consisting of alkyl, alkenyl, alkoxy, haloalkyl, haloalkoxy, halogen, hydroxy, mercapto, cyano, carboxy, alkoxycarbonyl, alkyloyloxy, acryloyloxy, methacryloyloxy, alkoxyalkylene, glycidoxy, formyl, phenyl, phenoxy, phenylalkoxy, phosphono, dialkylphosphono, phosphonooxy, dialkylphosphonooxy, alkylsulfonyl, phenylsulfonyl, heteroaryl, heteroaryloxy, heteroarylalkoxy, heterocyclyl, heterocyclylalkylenyl, and in the case of alkyl, alkenyl, alkynyl, and heterocyclyl, oxo and epoxy;
R₃ and R₄ are independently selected from the group consisting of HO—, alkyl-O—, (meth)acryloyl-O-alkylene-O—, phenyl-O—, heteroaryl-O—, and (M⁺)⁻O—; wherein M⁺ is a monovalent metal cation; and
wherein the hardenable material comprises a resin system.

24. The method of claim 23, wherein R₁ and R₂ are each independently alkyl, acryloyloxyalkylenyl, methacryloyloxyalkylenyl, glycidoxyalkylenyl, or glycidoxyphenyl.

25. The method of claim 23, wherein R₃ and R₄ are each independently alkoxy.

26. The method of claim 25, wherein R₁ and R₂ are methyl and R₃ and R₄ are ethoxy.

27. The method of claim 23, wherein the fluorescence has chromaticity coordinates x and y, wherein x is in the range of 0.13 to 0.19, and y is in the range of 0.05 to 0.22.

28. The method of claim 27, wherein x is in the range of 0.14 and 0.18, and y is in the range of 0.09 and 0.20.
